(19) United States
(12) Patent Application Publication
(10) Pub. No.: US 2007/0051035 A1
(43) Pub. Date: Mar. 8, 2007

(54) Kit containing meal components

(76) Inventors: Paola Giavedoni, Vlaardingen (NL); Fiona Clare McCurrach, Shambrook (GB); Jan Marcel van der Vaart, Vlaardingen (NL)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/568,561

(22) PCT Filed: Jul. 23, 2004
(86) PCT No.: PCT/EP04/08254

§ 371 Date: Feb. 17, 2006

(30) Foreign Application Priority Data
Aug. 19, 2003 (EP) 03077665.2

(51) Int. Cl. A01H 3/00; A61K 36/53; A61K 36/537; A61K 36/534

(52) U.S. Cl. 47/58.1 R; 424/773; 424/725; 424/745; 424/746; 424/747

(57) ABSTRACT

A method suited for prolonging freshness of a living, edible plant which is packed in a meal kit with at least one other meal ingredient or component, characterized in that the plant's roots remain in effective contact with a moisture donating source, such that during storage of the meal kit for several days the moisture source can donate to the plant or plant part enough of an aqueous fluid to ensure preservation of its freshness.

### TECHNICAL FIELD

The invention-deals with a package containing separate meal components, a so-called meal kit, particularly a kit comprising herbs as a meal ingredient.

### BACKGROUND AND PRIOR ART

A meal kit, as exemplified in e.g. EP1186541, consists of a package which contains several, at least two, components or ingredients which are meant for the preparation of a meal. Such meal may consist of a single dish or of several dishes. The kit ingredients may be ready for eating or may need some additional handling such as cooking, frying and/or stirring. Often for the meal preparation some ingredients are needed, such as eggs or milk, which are not present in the meal kit. The ingredients and components are present as separate kit items, packed or non-packed, just as their composition or consistency requires.

By minimizing time for shopping and cooking, meal kits provide primarily a commodity benefit for the busy consumer. Looking for separate meal ingredients needing visits of several shops becomes redundant. The ingredients for the meal are already selected, washed (vegetables), sometimes precooked and provided in weighted portions. The meal kit enables a quick and easy preparation of a meal which nevertheless may be tasty and healthy.

Traditionally, meal kits are composed with non-perishable ingredients having a long shelf life. However, the demand of fresh meal kits is growing. Fresh means that the kit contains fresh ingredients comprising vegetables, herbs, meat and/or fish and consequently has a short shelf life. Depending on the most sensitive ingredient shelf life varies from a few days to a few weeks. When fresh, perishable ingredients are present, chilled storage of the meal kit is needed.

The presence of fresh herbs in a dish often is much appreciated because herbs highly contribute to appearance and flavour of the dish. Fresh herbs, however, often are not available in the average supermarket and should be bought in a further shop and often in an undesired excess quantity. Often fresh herbs need same day processing, because most are known to deteriorate quickly.

For this reason, when herbs are included in a meal kit, they are only in dried form, which detracts from their desired contribution to the aroma and appearance of the prepared dish.

### SUMMARY OF THE INVENTION

We have developed a meal kit comprising at least two separate items, characterized in that at least one of the items consists of a living, edible plant provided with roots, which roots are in effective contact with a moisture donating source.

Effective contact means that the roots can absorb enough moisture from the moisture donating source to ensure the plant's freshness until the end of the shelf life of the meal kit.

### DESCRIPTION OF THE FIGURES

FIG. 1 shows a meal kit according to the invention consisting of a secondary packaging (a) containing several items (b) with a primary packaging (c) including a living plant (d) which is rooted in a growth medium (e).

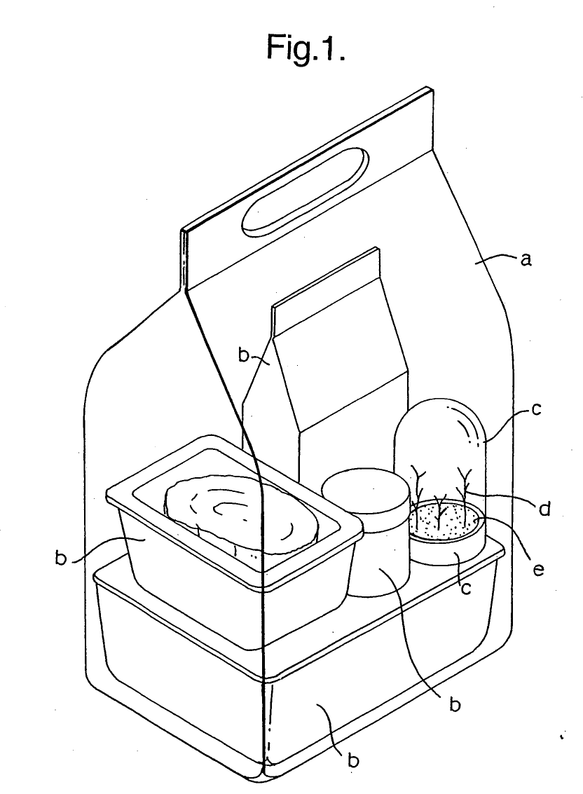

FIG. 2 shows an example how a living plant can be included in a meal kit (FIG. 2a). FIG. 2b shows the two parts of the plant package: a bottom part provided with a growth medium wherein seeds which can germ and grow in the medium. The upper part of the package acts like a little greenhouse which helps to protect the plant against pressure and squeezing damage (FIG. 2c). The consumer uses these as fresh ingredients for the meal by opening the plant container and separating the plant from the growth medium by cutting or pulling the stem (FIG. 2d).

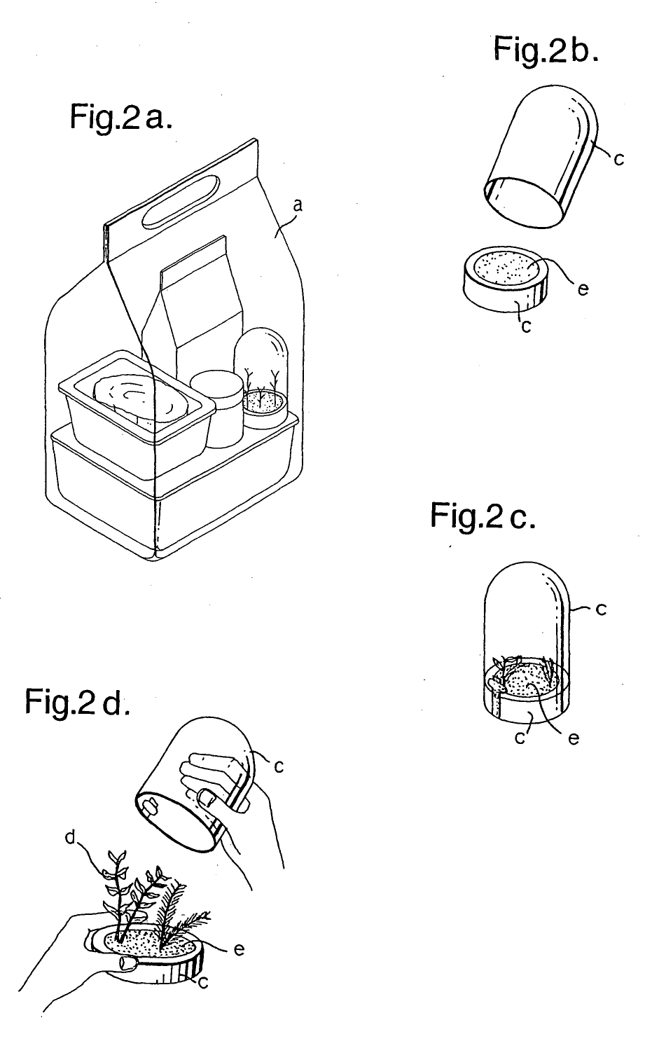

### DETAILS OF THE INVENTION

Chilled storage has caused a revolution in the food distribution chain. Fresh food, including vegetables, meat and fish can be transported over long distances without significant loss of quality and can be stored subsequently in chilled shop cabinets for a substantial period.

In the context of the present specification the condition denoted as plant freshness should be understood as the plant having substantially the same outer appearance as a healthy plant before it was removed from the place where it lived in nature or where it was grown in culture. Both U.S. Pat. Nos. 6,138,828 and 2003/0024163 illustrate some attempts to preserve freshness of plants. Those documents describe plants of which the roots are cut.

Some vegetable matter, particularly herbs, are more sensitive for deterioration than others and quickly loose their fresh appearance, even under chilled conditions. Freshly cut basil is a herb which is widely used in and which is typical for the Italian kitchen. When fresh basil is not available, the herb is used only in dried or frozen form for cooking. For fresh basil chilled storage is even detrimental: it appears to wither quickly and to obtain a dark coloration already at the same day.

Generally, a plant can better withstand unfavorable storage conditions when it still contains its roots with which it is able to replenish from an outside moisture source the water which is continuously evaporating from the plant leaves.

For this reason, outside nature plants are kept in pots or trays filled with ordinary soil. Usually the soil's moisture content is sufficient for leaving the plants without watering care during several days. US 2003/0024163 shows a closable tray in which e.g. herb plants can grow and be stored for several days without such care.

However, to include a pot with soil containing a living plant in a meal kit has never been proposed. Plant soil, being food incompatible, causes an uncontrollable health risk, because it may spill from the pot and the mess may pollute the kits content. It seems to be an obviously impractical measure. Therefore packing living plants, particularly fresh herbs, in meal kits, has escaped consideration and actual application.

We, however, have faced and overcome said objections and a possible prejudice against the use of living plants as a meal kit ingredient. We have found that a living plant can be hygienically and conveniently packed in a meal kit, while retaining its fresh appearance and flavouring quality for a substantial storage period.

It has been found essential for the survival of the plant when being part of the meal kit that its roots remain in effective contact with a moisture donating source, also denoted as moisture donating growth medium. Additional to ordinary plant soil, a hygienic alternative substrate can serve as a moisture donating source. Such alternative substrate comprises artificial plant growth mediums, preferably sterilized aqueous gels (e.g. agar gel) and sterilized spongy, porous materials such as porous stone (e.g. rock wool) and foamed polymers, which are valued because they are able to keep a proper amount of water absorbed for sustaining the plant life at least several days and because the plant becomes easily rooted in the substance. These materials are already used in distant arts, e.g. as solid substrate in modern greenhouses and in making flower arrangements (known e.g. under the name OASIS™). In contrast to present plants in pots with soil, plants rooted in such sterilized growth media like agar gel and rock wool provide an acceptable user friendly meal kit component which is fully food-grade. When preparing a dish the plant can be easily separated from the substrate by pulling or cutting without any messing of growth medium.

A sterilization treatment of the moisture donating source material is carried out, preferably after it has been soaked with moisture.

A preferred method for producing a plant which is attached to a moisture donating source is by placing a plant seed or some plant seeds in a piece of sterilized moisture donating growth medium and allowing the seed to germ and the plant to grow for about 2-3 weeks. The effective contact of the roots of the grown plant with the moisture donating source ensures an uninterrupted flow of moisture to the plant. So the freshness of the plant when eventually packed in the meal kit is prolonged considerably without giving special care. The preferred chilled storage of the meal kit of the invention provides the additional benefit that the outgrowth of the plant stops.

The moisture itself consists of an aqueous fluid having a composition which is commonly applied in the art of plant growing. This aqueous fluid may be plain water, but for the plant's initial growth and for further extending the plant's freshness period, it may contain beneficial plant nutrients and/or agents for suppressing growth of any undesired microorganisms. A suitable recipe for an aqueous fluid which contains supporting nutrients is contained in Table I

TABLE I

| | mg dissolved |
|---|---|
| | ingredient per |
| Dissolved ingredients | liter fluid |
| Potassium nitrate | 383 |
| Iron EDTA (13 wt. %)* | 14.3 |
| Ammonium nitrate | 38.3 |
| Magnesium sulphate | 193 |
| Monopotassium phosphate | 169 |
| Manganous sulphate (31 wt %)* | 1.44 |
| Copper sulphate (25 wt. %)* | 0.193 |
| Zinc sulphate (28 wt. %)* | 1.06 |
| Sodium molybdate (39 wt. %)* | 0.116 |

*ingredients which are added tot the fluid as an aqueous solution with indicated wt. concentration

The moisture donating source is effectively enclosed in a watertight container allowing only protruding the stem of the plant with its leaves through a narrow opening. The amount of moisture absorbed in the container is balanced such that leakage from the container in any position can not occur. Leakage is prevented further by an effective wrapping of the moisture source, e.g. with cling-film.

Besides the living plant, the meal kit may contain the usual ingredients and components which are present in a meal kit, fresh ingredients as well as non perishable ones. The meal kit of the invention usually contains fresh ingredients, comprising products from the group consisting of meat, fish, vegetables, herbs, fruits, sauces and fresh pastas and noodles. Therefore it needs to be stored under chilled conditions, which means storage at an average temperature between about +4° C. and about +10° C.

The living, edible plant may comprise any vegetables, rucola, fruits and onions, but preferably consists of one or more herbs, preferably herbs selected from the group consisting of parsley, basil, marjoram, oreganum, mint, thyme, bay, dill, mace, sage, sorrel, fennel and cress. Basil is a herb preferred for the preparation of Mediterranean style dishes.

In order to provide the kit plant with a mechanical protection against pressing and squeezing damage, it is preferably wrapped, more preferably surrounded by stiff, preformed packaging material, e.g. by a thermoformed polyethylene polyester plastic casing.

For the overall package of the meal kit one of the usual meal kit packaging solutions is selected. Form and substance are dictated by the kit's items and by wanted functionality and attractive presentation. Some kits have the form of a tray with recesses for each ingredient and are sealed by a transparent cover sheet. Alternative packages consist of a rigid box or of a more or less flexible bag. The kit items may be separately packed, each according to its typical composition and consistency.

The package of the meal kit preferably has a transparency which allows exposure of the living plant to life sustaining light as well as allowing visual inspection of the kit's contents and its presumed freshness by the customer.

The inexperienced customer is instructed, when preparing, the meal, to neatly separate the plant from the moisture donating source which is not meant for consumption. The amount of plant material is properly attuned to what is necessary for preparing the dish or the meal, so that waste is prevented.

The invention also comprises a method for prolonging freshness of a living, edible plant which is packed with at least one other meal ingredient or component in a meal kit, characterized in that the plant's roots remain in effective contact with a moisture donating source, such that during storage of the meal kit the moisture source can donate to the plant or plant part enough of an aqueous fluid to ensure preservation of the plant's freshness.

It has been shown that the present invention allows the use of living plants in meal kits, even herbs which are known as being very prone to withering under chilled conditions such as basil. For such sensitive plants a fresh appearance can be retained for a shelf life up to 7 days at 2° C.

The attached drawings show a particular embodiment of the invention. They are included for illustration purposes only and may not be construed as limiting the invention.

1. A meal kit comprising at least two separate items, characterized in that at least one of the items consists of a living, edible plant provided with roots, which roots are in effective contact with a moisture donating source.

2. A meal kit according to claim 1, characterized in that the plant consists of one or more herbs, preferably selected from the group consisting of parsley, basil, marjoram, oreganum, mint, thyme, bay, dill, mace, sage, sorrel, fennel and cress.

3. A meal kit according to claim 1, characterized in that the plant consists of basil.

4. A meal kit according to claim 1, characterized in that the moisture donating source is selected from the group consisting of natural plant soil or artificial plant growth mediums, preferably consisting of aqueous gels and, spongy, porous materials such as porous stone and foamed polymers and which source is soaked with enough aqueous fluid to sustain the plant life for several days.

5. A meal kit according to claim 1, characterized in that a sterilized moisture donating source is used.

6. A meal kit according to claim 1, characterized in that the moisture donating source is effectively enclosed in a watertight container allowing only protruding the stem of the plant with its leaves through a narrow opening.

7. A meal kit according to claim 1, characterized in that the kit's packaging material has a transparency which allows exposure of the plant to light and which allows visual inspection of the plant's freshness.

8. A method suited for prolonging freshness of a living, edible plant which is packed in a meal kit with at least one other meal ingredient or component, characterized in that the plant's roots remain in effective contact with a moisture donating source, such that during storage of the meal kit for several days the moisture source can donate to the plant or plant part enough of an aqueous fluid to ensure preservation of its freshness.
